(19) United States
(12) Patent Application Publication
(10) Pub. No.: US 2013/0289468 A1
(43) Pub. Date: Oct. 31, 2013

(54) LAPAROSCOPIC APPLICATOR AND USES THEREOF

(71) Applicant: AFCELL MEDICAL, Parsippany, NJ (US)

(72) Inventor: Robin R. YOUNG, Wayne, PA (US)

(73) Assignee: AFCELL MEDICAL, Parsippany, NJ (US)

(21) Appl. No.: 13/804,785

(22) Filed: Mar. 14, 2013

Related U.S. Application Data
(60) Provisional application No. 61/638,272, filed on Apr. 25, 2012.

(51) Int. Cl. A61F 13/00

(52) U.S. Cl. CPC A61F 13/00068
USPC 604/11

(57) ABSTRACT

An applicator for use in a laparoscopic surgery is described. The applicator allows the effective delivery of a surgical dressing, such as an allograft having at least one layer of human amnion and chorion tissues, to the surgical site, to thereby improve the performance of the surgery, e.g., by reducing adhesions, scar formation while also reducing inflammation and risk of post-operative infection.

### CROSS-REFERENCE TO RELATED APPLICATIONS

This application is entitled to priority pursuant to 35 U.S.C. §119(e) to U.S. Provisional Patent Application No. 61/638,272, filed Apr. 25, 2012, which is hereby incorporated by reference herein in its entirety.

### BACKGROUND OF THE INVENTION

1. Field of Invention

Embodiments of the present invention relate to methods and products for improving laparoscopic surgery. In particular, embodiments of the present invention relate to an applicator for applying a surgical dressing through a cannula for subsequent covering of the surgical site during a laparoscopic surgery.

2. Background of the Invention

Laparoscopic surgery, also referred to as minimally invasive surgery (MIS), is a surgical procedure with the assistance of a video camera and several thin instruments, during which, small incisions, e.g., of up to half an inch, are made and plastic tubes, ports or cannulae, are placed through these incisions. The camera and the instruments are then introduced through the tubes which allow access to the inside of the patient.

U.S. Patent Application Publication No. U.S.20120035743, the content of which is herein incorporated by reference, describes constructs comprising an allograft having at least one layer of amnion and chorion tissues for use during minimally invasive surgeries. The constructs are adapted for the ease of insertion through a cannula for subsequent covering of the surgical site during a minimally invasive surgery.

The amnion is a thin, cellular, extraembryonic collagen matrix that forms the inner membrane of a closed placental sac surrounding and protecting an embryo in reptiles, birds, and mammals. The sac contains the fetus and amniotic fluid or liquor amnii, in which the embryo is immersed, nourished and protected. Amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid.

The amnion has multiple functions, e.g., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and muscle repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograph for repairing corneal defects. See, for example, Tsai and Tseng, Cornea. 1994 September;13(5):389-400; and Dua et al., Br. J. Ophthalmol 1999, 83:748-20 752. In addition, amnion and amniotic fluid have recently been used as sources of placental stem cells. See, e.g., U.S. Pat. No. 7,255,879 and WO 200073421.

The present invention relates to methods and products for improving laparoscopic surgery. In particular, embodiments of the present invention relate to an applicator for applying a surgical dressing, preferably a surgical dressing comprising an allograft having at least one layer of amnion and chorion tissues, to a surgical site during a laparoscopic surgery.

### BRIEF SUMMARY OF THE INVENTION

In one general aspect, the present invention relates to an applicator for use in a laparoscopic surgery, the applicator comprising:
a liquid conduit;
a bulb connected to the liquid conduit; and
a surgical dressing placed over at least a portion of the bulb and an end of the liquid conduit connected to the bulb,
   wherein the surgical dressing has a size and shape appropriate for covering a surgical site; and at least one of the portion of the bulb and the end of the liquid conduit connected to the bulb comprises a porous mesh material that allows the application of a liquid from the liquid conduit to the surgical dressing to thereby apply the surgical dressing to the surgical site.

In another general aspect, the present invention relates to a method of preparing an applicator for use in a laparoscopic surgery, the method comprising:
obtaining a liquid conduit;
obtaining a bulb;
connecting the bulb to an end of the liquid conduit;
obtaining a surgical dressing having a size and shape appropriate for covering a surgical site; and
placing the surgical dressing over at least a portion of the bulb and the end of the liquid conduit connected to the bulb,
   wherein at least one of the portion of the bulb and the end of the liquid conduit connected to the bulb comprises a porous mesh material that allows the application of a liquid from the liquid conduit to the surgical dressing to thereby apply the surgical dressing to the surgical site.

Another general aspect of the present invention relates to an improved laparoscopic surgery. The improvement comprises inserting an applicator according to an embodiment of the present invention into the small incision and the cannula employed in the laparoscopic surgery; and applying a liquid from the liquid conduit to the surgical dressing to thereby apply the surgical dressing to the surgical site.

Preferably, the surgical dressing comprises an allograft having at least one layer of human amnion and chorion tissues.

In a preferred embodiment of the present invention, the human amnion and chorion tissues and amniotic fluid are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

Another general aspect of the present invention relates to a kit, comprising:
(1) an applicator comprising a liquid conduit and a bulb connected to a liquid conduit;
(2) a plurality of surgical dressings wherein at least two of the surgical dressings are different in at least one of size and shape; and
(3) instructions for applying the surgical dressing to a surgical site with the applicator.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates a laparoscopic applicator according to an embodiment of the present invention, A: the applicator without the surgical dressing; and B: the complete applicator with the surgical dressing; and

FIG. 2 illustrates a laparoscopic applicator according to another embodiment of the present invention, A: the applicator without the surgical dressing; and B: the complete applicator with the surgical dressing.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

Embodiments of the present invention relate to an applicator for use in a laparoscopic surgery, the applicator comprising:
a liquid conduit;
a bulb connected to the liquid conduit; and
a surgical dressing placed over at least a portion of the bulb and an end of the liquid conduit connected to the bulb,
   wherein the surgical dressing has a size and shape appropriate for covering a surgical site; and at least one of the portion of the bulb and the end of the liquid conduit connected to the bulb comprises a porous mesh material that allows the application of a liquid from the liquid conduit to the surgical dressing to thereby apply the surgical dressing to the surgical site.

In one embodiment according to the present invention, the bulb is liquid impermeable, the end of the liquid conduit connected to the bulb comprises a porous mesh material that allows the application of a liquid from the liquid conduit to the surgical dressing to thereby apply the surgical dressing to the surgical site, and the surgical dressing is placed over the bulb and the end of the liquid conduit connected to the bulb.

In another embodiment according to the present invention, the bulb is liquid permeable. At least a portion of the bulb comprises a porous mesh material that allows the application of a liquid from the liquid conduit to the bulb, then the surgical dressing to thereby apply the surgical dressing to the surgical site. Preferably, a top portion of the bulb comprises the porous mesh material that allows the application of a liquid from the liquid conduit to the top portion of the bulb, then to the surgical dressing to thereby apply the surgical dressing to the surgical site. Other portions of the bulb can also comprise the porous mesh material for the application of the liquid to the surgical dressing placed over the portion of the bulb that comprises the porous mesh material.

The liquid conduit and the bulb can be made of any surgical grade materials, e.g., disposable polymers, such as polyethylene or polyetheretherketone (PEEK) polymer. They can be made of the same material or different materials.

The porous mesh material can be any porous mesh material suitable for surgery, such as one selected from the group consisting of any medical grade polymer or monomer, including but not limited to, polyvinyl alcohol (PVA), polytetrafluroethylene (PTFE), expanded polytetrafluroethylene (ePTFE), porous polyethylene, porous polypropylenes, liquid polyurethanes for hollow-fiber implants, polyurethanes for dip-molding, biostable polyurethanes and thermoplastic polyurethanes.

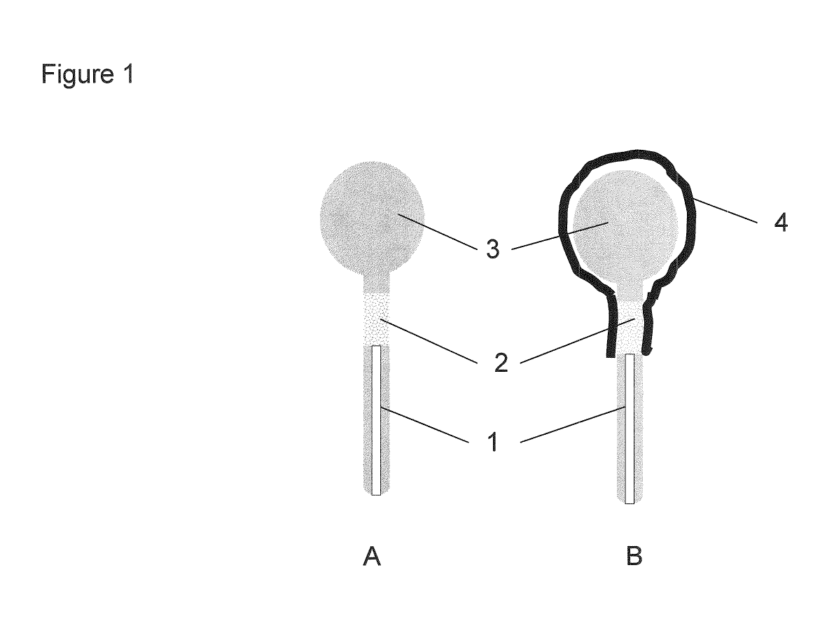

Referring to FIG. 1, according to an embodiment of the present invention, the applicator contains a liquid conduit (1), which has an end comprising a porous mesh material (2), and a liquid impermeable bulb (3), which is connected to the end of the liquid conduit (1). The bulb has a diameter that is sized for ease of insertion into a small incision and a cannula employed in the laparoscopic surgery, such as a diameter of about 3 mm. In one embodiment, the bulb is solid. Preferably, the bulb and the conduit are rigid or semi-rigid in order to facilitate the insertion of the applicator during or after an MIS. The applicator further comprises a surgical dressing (4) placed over the bulb (3) and the end of the liquid conduit connected to the bulb. The surgical dressing can be any dressing suitable for covering a surgical site resulting from a laparoscopic surgery. It has the size and shape appropriate for covering a surgical site. For example, the dressing can be spherical or semi-spherical, rectangular, etc.

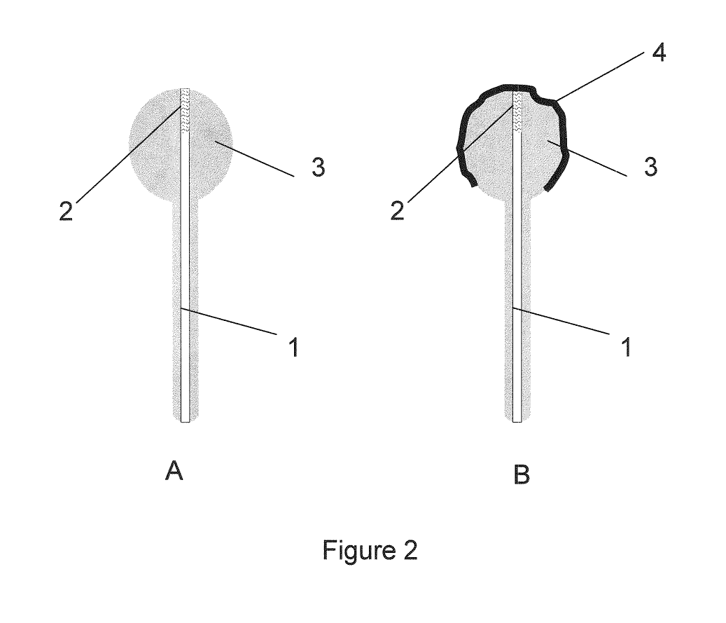

Referring to FIG. 2, according to another embodiment of the present invention, the applicator contains a liquid conduit (1) and a bulb (3) connected to the liquid conduit. The bulb is liquid permeable, a portion of which contains a porous mesh material (2). In one embodiment, the bulb is solid, except a portion of which that allows liquid connectivity with the liquid conduit. A part or whole of this liquid conductive portion can be made of or filled with the porous mesh material. The applicator further comprises a surgical dressing (4) placed over the liquid permeable portion of bulb (3).

The surgical dressing is physiologically compatible and can be made of any physiologically compatible material. In one embodiment of the present invention, the surgical dressing comprises an allograft. According to a preferred embodiment of the present invention, the surgical dressing comprises an allograft having at least one layer of amnion and chorion tissues.

The allograft can have one or more layers of amnion or one or more layers of chorion, or have a combination of multiple layers of amnion and multiples layers of chorion. The multiple layers of amnion and chorion can be in any combination arranged in any order. The multiple layers in the allograft can be subject to a cross-linking treatment to make the layers closely adhere to each other in an integrated form.

An applicator according to an embodiment of the present invention allows for the application of a liquid to thereby apply a surgical dressing to a surgical site. Any liquid suitable for use in a surgical procedure can be used. The liquid can be a sterile saline solution or other sterile fluid. The liquid can also be used to reduce adhesions, and scar formation while also reducing inflammation and risk of post-operative infection at the surgical site to which it is applied, such as, for example, amniotic fluid.

In one embodiment of the present invention, the surgical dressing can carry one or more therapeutic agents, such as growth enhancing agents, morphogenic proteins, small molecule compounds, pharmaceutical agents, anti-microbial agents, anti-inflammatory agent, agents that prevent scarring, adhesions and tethering of internal tissue at or near the surgery site, analgesics, etc., to further improve the performance and reduce the complications of MIS. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing. In another embodiment, the liquid used to apply the surgical dressing can carry one or more therapeutic agents in view of the present disclosure.

An applicator according to an embodiment of the present invention can be used for any laparoscopic surgery. Examples of laparoscopic surgery include, but are not limited to, laparoscopic biopsy, such as for an abdominal organ; laparoscopic surgery of abdominal organs such as an appendectomy, removal of colon polyps, and treatment of diverticulitis; laparoscopic surgery for the treatment of gynecological conditions such as endometriosis, ectopic pregnancy, hysterectomy, infertility and tubal ligation, or a myomectomy to remove uterine fibroids; laparoscopic surgery for the treatment or repair of torn cartilage, and ligaments, and repair of herniated spinal disks; laparoscopic surgery for the correction of abnormalities in the paranasal sinuses that cause chronic sinus infections; and laparoscopic surgery for urological organs such as for the removal of kidneys, prostates and lymph nodes.

The surgical dressing can be any size and shape suitable for covering the surgical site to which it will be applied and will depend on the particular laparoscopic surgery being performed. The surgical dressing can be, for example, rectangular, triangular, cylindrical, donut shaped, tubular shaped, parabolic, or arc shaped. As an illustrative example, in a laparoscopic myomectomy to remove uterine fibroids, the surgical dressing is preferably has multiple adjacent parabolic shapes, which allows for the construct to unfold around tubes or other adjacent structures to thereby better localize the surgical dressing to a desired position during the laparoscopic surgery.

In one embodiment of the present invention, one or more corners of the surgical dressing, such as the allograft, are rounded or flattened to prevent the corners from catching during application to the surgical site. In view of the present disclosure, any method known to those skilled in the art can be used to make the corners of the construct or allograft round or flatten.

Another aspect of the present invention relates to a method of preparing an applicator for use in a laparoscopic surgery, the method comprising:
obtaining a liquid conduit;
obtaining a bulb;
connecting the bulb to the liquid conduit;
obtaining a surgical dressing having a size and shape appropriate for covering a surgical site; and
placing the surgical dressing over at least a portion of the bulb and the end of the liquid conduit connected to the bulb,
   wherein at least one of the portion of the bulb and the end of the liquid conduit connected to the bulb comprises a porous mesh material that allows the application of a liquid from the liquid conduit to the surgical dressing to thereby apply the surgical dressing to the surgical site.

In one embodiment of the present invention, the surgical dressing is separately packaged before it is assembled together with the bulb and the connected liquid conduit. The surgical dressing can be dried over a frame, such as a polymer mesh frame or a stainless steel frame, into the appropriate shape. The dried surgical dressing can then be removed from the frame, retaining the shape of the frame, and subsequently packaged. The frame can be any shape. Prior to use, the surgical dressing is wet by sterile saline or another sterile fluid applied to the dressing. The wet dressing collapses over the bulb (3) and the connected porous end (2) of the conduit to result in an applicator as that illustrated in FIG. 1B, or over the porous portion of bulb (3) to result in an applicator as that illustrated in FIG. 2B.

In another embodiment of the present invention, a wet surgical dressing is placed over the bulb and dried, then packed together with the bulb and the connected liquid conduit. Prior to use, the surgical dressing is wet by sterile saline or another sterile fluid applied to the dressing by, for example, applying a liquid to the surgical dressing from the liquid conduit.

Another general aspect of the present invention relates to an improved method of performing an MIS. The improvement comprises inserting an applicator according to an embodiment of the present invention into the small incision and the cannula employed in the laparoscopic surgery; and applying a liquid from the liquid conduit to the surgical dressing to thereby apply the surgical dressing to the surgical site. Any liquid suitable for use in a surgical procedure, such as a sterile saline or other sterile solution, can be applied to the liquid conduit. The liquid is released from the porous end of the conduit and hydrates the surgical dressing in a manner that allows it to unfold in vivo at the surgical site and assume the form of a flat sheet, or other appropriate shape, to thereby cover the surgical site.

In a preferred embodiment, the surgical dressing comprises an allograft having at least one layer of amnion and chorion tissue. When the allograft has multiple layers of amnion and chorion tissue, the surgical dressing can be applied to a surgical site according to an improved method of the present invention with either tissue directly in contact with the surgical site. In addition amnion tissue has two surfaces: (1) an outer surface in contact with chorion tissue; and (2) an inner surface in contact with amniotic fluid. Likewise, chorion tissue also has two surfaces: (1) an outer surface that is contact with maternal cells; and (2) and inner surface that is in contact with amnion tissue. According to embodiment of the present invention, either surface (ie. inner or outer) of either tissue of the allograft (ie. amnion or chorion) can be placed in direct contact with the surgical site to which it is applied.

The improvement can be applied to any procedure of MIS in view of the present disclosure. The circumference of the surgical dressing, such as one comprising an allograft, can be larger than the surgical site it will be implanted on so that when hydrated it will fully encase the surgical site. The circumference of the surgical dressing can also be the same size as the surgical site it will be implanted on.

An improved method of performing an MIS according to embodiments of the present invention can result in reduced inflammation, scarring, or adhesions, or a decreased risk of infection at the surgical site post-surgery.

In another embodiment of the present invention, a surgical dressing comprising an allograft having at least one layer of amnion and chorion tissues is used to cover a skin incision resulting from the MIS. The allograft patch can be of any size suitable for covering the sutures or other type of tissue injuries at the site of skin incision.

Preferably, a relatively thick layer of allograft is used to cover the skin incision. In one embodiment of the invention, the allograft patch has a thickness of about 2 mm to 4 mm. According to embodiments of the present invention, the allograft for covering the skin incision can have one layer of amnion or one layer of chorion, it can have multiple layers of amnion or multiple layers of chorion, or a combination of multiple layers of amnion and chorion in any combination of amnion and chorion. When the allograft has multiple layers of amnion and multiple layers of chorion, the layers can be arranged in any order, and the allograft can be applied to the skin incision with either amnion tissue or chorion tissue in direct contact with the skin.

In another embodiment of the present invention, amniotic fluid can be applied into the small incision or the cannula employed in the MIS to thereby cover the surgical site or skin incision with the amniotic fluid. The amniotic fluid can also be applied to cover a skin incision resulting from the MIS. The amniotic fluid can be used as the liquid to hydrate and deliver the surgical dressing to a surgical site according to an embodiment of the present invention.

The amniotic fluid and the construct can be applied individually or in combination during the surgery. The amniotic fluid can be processed so that it has a relatively high or low viscosity for ease of application. Methods known to those skilled in the art can be used to prepare amniotic fluid with a desirable viscosity in view of the present disclosure.

There are several attributes which make an allograft having at least one of amnion and chorion tissues a preferred material for use in an MIS. Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used in an MIS.

Human allograft amnion and chorion have the ability to prevent scarring, reduce inflammation, inhibit microbial infection and improve healing. During an MIS, the surgeon is required to work in very tight spaces. Covering the surgical site with a flat sheet of membrane that improves healing can be extremely difficult for the surgeon. By creating and using a collapsible and expandable construct, allografts of human anmion and/or chorion can now be delivered through the cannula to allow subsequently covering of the surgical site with ease. The allografts have the ability to reduce adhesions, scar formation while also reducing inflammation and risk of post-operative infection.

Amnion, chorion and amniotic fluid used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other forms of supporting material, such as a frame, which would facilitate the subsequent manipulation of the amnion, such as sterilizing, sizing, cataloging, and shipping of the amnion.

The present invention encompasses a kit comprising an applicator for use in an MIS or other type of surgery according to an embodiment of the present invention and instructions on how to use the applicator in the MIS. An applicator with any surgical dressing for use in a surgery, such as an MIS, according to embodiments of the present invention can be included in the kit. Preferably, the surgical dressing comprises an allograft comprising at least one layer of human amnion and chorion tissues. The applicator is adapted for insertion into a small incision and a cannula employed in the minimally invasive surgery for access to the surgical site, and the surgical dressing has a shape appropriate for covering the surgical site.

In a preferred embodiment, the kit comprises a plurality of surgical dressings for MIS, and at least two of the plurality of dressings have different shapes or sizes suitable for covering different surgical sites. The surgical dressing in the construct can further comprise one or more therapeutically active agents, such as anti-microbial agents, growth enhancing agents, anti-inflammatory agents, analgesics, etc.

According to an embodiment of the present application, the kit further comprises a liquid for use with an applicator of the present invention for a surgical dressing to a surgical site. In a preferred embodiment, the kit comprises an amniotic fluid and instructions on how to use the amniotic fluid in the minimally invasive surgery.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. An applicator for use in a laparoscopic surgery, the applicator comprising:
a liquid conduit;
a bulb connected to the liquid conduit; and
a surgical dressing placed over at least a portion of the bulb and an end of the liquid conduit connected to the bulb,
wherein the surgical dressing has a size and shape appropriate for covering a surgical site; and at least one of the portion of the bulb and the end of the liquid conduit connected to the bulb comprises a porous mesh material that allows the application of a liquid from the liquid conduit to the surgical dressing to thereby apply the surgical dressing to the surgical site.

2. The applicator of claim 1, wherein the surgical dressing comprises an allograft having at least one layer of human amnion and chorion tissues.

3. The applicator of claim 1, wherein the bulb and the liquid conduit are made of the same or different surgical grade materials.

4. The applicator of claim 1, wherein the bulb and the liquid conduit are rigid or semi-rigid.

5. The applicator of claim 1, wherein the porous mesh material is selected from the group consisting of any medical grade polymer and monomer.

6. The applicator of claim 1, wherein the porous mesh material is selected from the group consisting of polyvinyl alcohol (PVA), polytetrafluroethylene (PTFE), expanded polytetrafluroethylene (ePTFE), porous polyethylene, porous polypropylenes, liquid polyurethanes for hollow-fiber implants, polyurethanes for dip-molding, biostable polyurethanes and thermoplastic polyurethanes.

7. The applicator of claim 1, wherein the surgical dressing comprises one or more therapeutic agents to further improve the performance and reduce the complications of the laparoscopic surgery.

8. A method of preparing an applicator for use in a laparoscopic surgery, the method comprising:
obtaining a liquid conduit;
obtaining a bulb;
connecting the bulb to the liquid conduit;
obtaining a surgical dressing having a size and shape appropriate for covering a surgical site; and
placing the surgical dressing over at least a portion of the bulb and an end of the liquid conduit connected to the bulb,
wherein at least one of the portion of the bulb and the end of the liquid conduit connected to the bulb comprises a porous mesh material that allows the application of a liquid from the liquid conduit to the surgical dressing to thereby apply the surgical dressing to the surgical site.

9. The method of claim 8, wherein the surgical dressing comprises an allograft having at least one layer of human amnion and chorion tissues.

10. The method of claim 9, further comprising drying the allograft over at least the portion of the bulb and the end of the liquid conduit connected to the bulb.

11. The method of claim 9, wherein the human amnion and chorion tissues are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

12. In an improved method of performing a laparoscopic surgery, the improvement comprising:
inserting an applicator of claim 1 into the small incision and the cannula employed in the laparoscopic surgery; and
applying the liquid from the liquid conduit to the surgical dressing to thereby apply the surgical dressing to a surgical site.

13. In the method of claim 12, wherein the surgical dressing comprises an allograft having at least one layer of human amnion and chorion tissues.

14. In the method of claim 12, wherein improvement further comprises applying an amniotic fluid to the surgical site

15. A kit comprising:
(1) an applicator comprising a liquid conduit and a bulb connected to a liquid conduit;
(2) a plurality of surgical dressings wherein at least two of the surgical dressings are different in at least one of size and shape; and
(3) instructions for applying the surgical dressing to a surgical site with the applicator.

16. The kit of claim 15, further comprising an amniotic fluid and instructions on applying the amniotic fluid to cover the surgical site.

17. The kit of claim 15, wherein at least one of the surgical dressing comprises an allograft having at least one layer of human amnion and chorion tissues.

18. The kit of claim 15, wherein at least one of a portion of the bulb and an end of the liquid conduit connected to the bulb comprises a porous mesh material that allows the application of a liquid from the liquid conduit to the surgical dressing placed over the portion of the bulb or the end of the liquid conduit to thereby apply the surgical dressing to a surgical site.

19. The kit of claim 18, wherein the porous mesh material is selected from the group consisting of polyvinyl alcohol (PVA), polytetrafluroethylene (PTFE), expanded polytetrafluroethylene (ePTFE), porous polyethylene, porous polypropylenes, liquid polyurethanes for hollow-fiber implants, polyurethanes for dip-molding, biostable polyurethanes and thermoplastic polyurethanes.

20. The kit according to claim 15, further comprising an amniotic fluid.
